(19) United States
(12) Patent Application Publication
(10) Pub. No.: US 2024/0312327 A1
(43) Pub. Date: Sep. 19, 2024

(54) MONITORING SYSTEM FOR CONGREGATE CARE FACILITIES

(71) Applicant: LIFEGUARD DIGITAL HEALTH INC., Vancouver (CA)

(72) Inventors: Jeffery Les Hardy, Surrey (CA); Colton Calderone, North Vancouver (CA)

(21) Appl. No.: 18/575,179

(22) PCT Filed: Jun. 28, 2022
(86) PCT No.: PCT/CA2022/051032

§ 371 Date: Dec. 28, 2023

Related U.S. Application Data
(60) Provisional application No. 63/215,734, filed on Jun. 28, 2021.

(51) Int. Cl. G08B 21/04; G08B 25/01

(52) U.S. Cl. CPC G08B 21/043; G08B 21/0446; G08B 21/0453; G08B 21/0476; G08B 25/014

(57) ABSTRACT

A system for monitoring residents in a congregate care facility comprises administration devices. wearable devices. and room devices. A wearable device is associated with a resident and configured to determine an alert associated with the resident and to communicate the alert to the administration device. Each room device is associated with one of the residents. The room devices comprises a camera configured to communicate with the administration device, and is also configured to allow for voice communications with the administration device. The administration device is configured to receive the alert from the wearable device, identify the resident and the room device associated with the wearable device, activate the camera of the room device associated with the resident, and allow for communication between the room device associated with the resident and the administration device.

### FIELD OF THE INVENTION

The present invention relates to a system for monitoring elderly or physically or mentally challenged residents in congregate care facilities, and in particular, a system for detecting and alerting staff regarding such residents who may be in distress in congregate care facilities.

### BACKGROUND OF THE INVENTION

Residents in congregate living facilities, such as long-term care facilities or nursing homes, are typically elderly and/or physically or mentally challenged. For such residents, it is important that when they are in distress (such as from a fall or from some other condition), proper assistance is provided in a timely manner. This requires that appropriate monitoring of the residents be carried out, such that when a resident in possible distress is detected, appropriate staff are notified so that proper assistance may be rendered to the residents.

There is therefore a desire for a system that is able to monitor residents in congregate care facilities, subject to their consent and other privacy protections, and alert staff in the event that a resident may require medical assistance.

### SUMMARY OF THE INVENTION

In one embodiment, the invention is a system for monitoring residents in congregate care facilities to detect when they may be in need of assistance. The system comprises one or more wearable devices, one or more room devices, and one or more administration devices. Each of the wearable devices is removably attached to one of the residents and is associated with that resident. In addition, each of the wearable devices is also associated with a room in the facility (e.g., the room where the resident normally resides). The wearable device is configured to detect certain physiological events, such as a potential fall, irregular breathing, irregular heartbeat, or the like.

When a potential fall or some other abnormal physiological event is detected by the wearable device, the wearable device generates a prompt to the resident asking whether the resident has indeed suffered a fall. If the resident responds affirmatively (or if no response is provided), a fall alert is sent by the wearable device to the appropriate administration device. The fall alert may also be sent to other devices as well. There may be one administration device in each particular section of the facility. The administration device comprises a user interface that will display a suitable message to a staff member. In addition, the administration device may cause the room device associated with the wearable device to activate. Each of the rooms in the facility preferably has a room device. The room device may comprise a camera that, when appropriate resident consent is given, allows for video of the interior of the room to be sent to the administration device for display on the user interface.

The administration device is also able to initiate a call (phone or video) between the room device and the administration device to allow for communication between the staff member and the resident. The staff member is then able to determine whether the resident is in need of further assistance.

In one embodiment, a system for monitoring residents in a congregate care facility comprises one or more administration devices, one or more wearable devices, and one or more room devices. Each of the one or more wearable devices are removably attached to and associated with one of the residents and configured to determine an alert associated with the resident and to communicate the alert to at least one of the administration devices. Each of the room devices is associated with one of the residents. For the one or more room devices associated with one of the residents, at least one of the room devices comprises a camera configured to communicate with the administration device, and at least one of the room devices is configured to allow for voice communications with the administration device. The administration device is configured to: (i) receive the alert from the wearable device: (ii) identify the resident and the room device associated with the wearable device: (iii) activate the camera of the room device associated with the resident: and (iv) allow for communication between the room device associated with the resident and the administration device.

In another embodiment, the alert is a physiological alert. The physiological alert may be selected from one or more of the following: physical fall, blood pressure, heart rhythm, body temperature, heart rate, oxygenation level, and blood sugar.

In still another embodiment, each of the one or more wearable devices comprises one or more sensors configured to detect information relating to the physiological alert.

In still yet another embodiment, each of the one or more wearable devices is configured to communicate the alert when the information relating to the physiological alert detected by the one or more sensors fall outside of a predetermined range of values.

In another embodiment, the system further comprises a server. The server is configured to receive the alert from one of the wearable devices and is further configured to determine an appropriate ones of the administration devices to which to send the alert.

In still another embodiment, the determination of the appropriate ones of the administration devices to which to send the alert is determined, at least in part, by determining the administration device that is closest to the wearable device from which the alert was communicated.

In still yet another embodiment, each of the administration devices is associated with one or more of the residents.

In a further embodiment, the determination of the appropriate one of the administration devices to which to send the alert is determined, at least in part, by determining the administration devices associated with the resident whose wearable device from which the alert was communicated.

In another embodiment, a system for monitoring residents in a congregate care facility comprises one or more administration devices, one or more wearable devices, and one or more room devices. Each of the one or more wearable devices is removably attached to and associated with one of the residents and is configured to receive a request for assistance from with the resident and to communicate the request for assistance to at least one of the administration devices. Each of the room devices is associated with one of the residents. For the one or more room devices associated with one of the residents, at least one of the room devices comprises a camera configured to communicate with the administration device, and at least one of the room devices is configured to allow for voice communications with the administration device. The administration device is configured to (i) receive the request for assistance from the wearable device: (ii) identify the resident and the room device associated with the wearable device: (iii) activate the camera of the room device associated with the resident: and (iv) allow for communication between the room device associated with the resident and the administration device.

In still another embodiment, the system further comprises a server. The server is configured to receive the request for assistance from one of the wearable devices and is further configured to determine an appropriate ones of the administration devices to which to send the request for assistance.

In a further embodiment, the determination of the appropriate ones of the administration devices to which to send the request for assistance is determined, at least in part, by determining the administration device that is closest to the wearable device from which the request for assistance was communicated.

In still a further embodiment, the determination of the appropriate one of the administration devices to which to send the request for assistance is determined, at least in part, by determining the administration devices associated with the resident whose wearable device from which the request for assistance was communicated.

In another embodiment, a system for monitoring residents in a congregate care facility comprises a central server, one or more administration devices in communications with the central server, and one or more wearable devices in communications with the central server.

Each of the one or more wearable devices is removably attached to and associated with one of the residents and configured to determine a location of the wearable device and to communicate the location to the central server from time to time. The wearable device is configured to generate an alert when the wearable device determines that it is outside of a predefined geographical area, the alert being transmitted to the central server. The central server is configured to transmit the alert and the location of the wearable device from time to time to one or more of the administration devices.

In yet another embodiment, the central server is configured to transmit the alert and the location of the wearable device from time to time to the administration device that is closest to the wearable device.

In still yet another embodiment, each of the administration devices is associated with one or more of the residents.

In a further embodiment, the central server is configured to transmit the alert and the location of the wearable device from time to time to the administration device that is associated with the resident whose wearable device from which the alert was communicated.

The foregoing was intended as a summary only and a description of only some of the aspects of the invention. It was not intended to define the limits or requirements of the invention. Other aspects of the invention will be appreciated by reference to the detailed description of the preferred embodiments.

### BRIEF DESCRIPTION OF THE DRAWINGS

The invention will be described by reference to the detailed description of the embodiments and to the drawings thereof in which:

FIG. 1 depicts the invention in accordance with one embodiment:

FIG. 2 shows an embodiment of the wearable device:

FIG. 3 shows the components of the invention:

FIG. 4 shows another embodiment of the invention;

FIG. 5 is a flowchart showing the process flow for detecting a potential fall in accordance with one embodiment:

FIG. 6 is a flowchart showing the process flow for detecting when a resident is outside of a predefined geographical area in accordance with one embodiment:

FIG. 7 is a flowchart showing the process flow for a help alert sent by a resident in accordance with one embodiment; and

FIG. 8 depicts another embodiment of the invention.

### DETAILED DESCRIPTION OF THE INVENTION

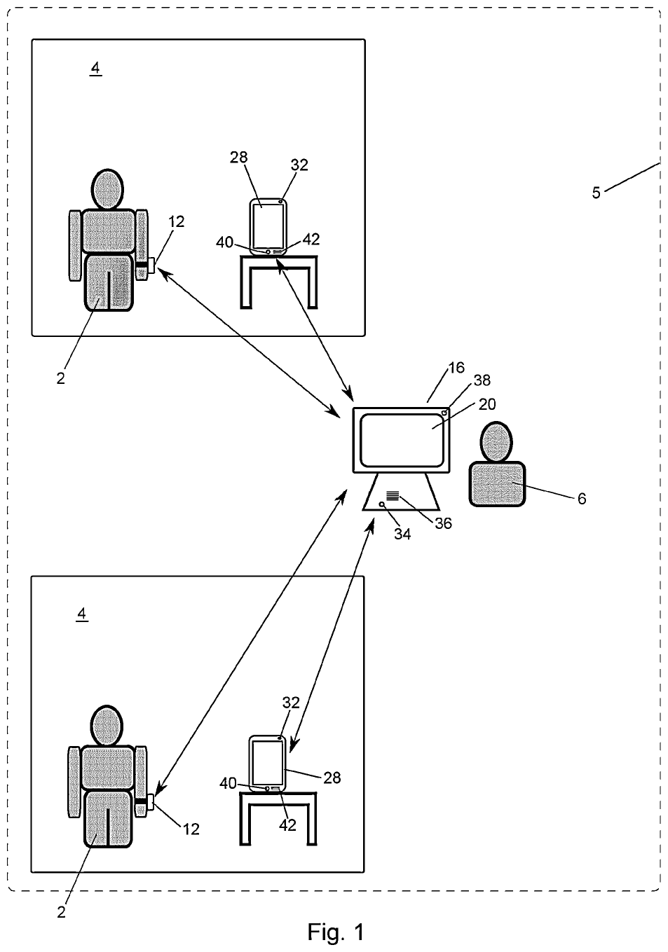

Referring to FIGS. 1 to 3, according to one embodiment of the invention, the system 10 is preferably used in congregate care facilities 5. Such congregate care facilities 5 may include nursing homes, long-term care facilities, hospitals, prisons, or the like. The system 10 comprises one or more wearable devices 12, each of which is configured to be removably attached to a resident 2 of the congregate care facility 5. The wearable device 12 may take the form of a watch, a bracelet, a neck chain, or some other device that may be worn by the resident 2. For example, in one embodiment, the wearable device 12 may take the form a smartwatch that may be worn on a wrist of the resident 2.

Each of the wearable devices 12 is preferably associated with one of the residents 2. This may be done through a registration process, whereby each of the wearable devices 12 is linked to one of the residents 2. In addition, the registration process may also associate each of the residents 2 with a room 4 of the congregate care facility 5. The room 4 associated with the resident 2 may be the room in which the resident 2 is normally located. Each of the wearable devices 12 preferably comprises a wearable device processing unit 14.

The system 10 further comprises one or more administration devices 16. For example, in the context of the congregate care facility 5, each of the administration devices 16 may be located at a front desk of a section of the congregate care facility 5 or at some other central location within the congregate care facility 5. Alternatively, the administration devices 16 may be located away from the congregate care facility 5 (e.g. at a remote location). Each of the administration devices 16 comprises an administration device processing unit 18 and an administration device interface 20. The administration device interface 20 may include a monitor or a screen. The administration device processing unit 18 is configured to enable communications between the administration device 16 and the wearable device 12, preferably through wireless communications. Similarly, the wearable device processing unit 14 is configured to enable communications between the wearable device 12 and the administration device 16, preferably through wireless communications. The wireless communication may be through Wi-Fi, ZigBee, Bluetooth, cellular networking, or the like. Where the administration devices 16 are located away from the congregate care facility 5, the communications between the administration devices 16 and the wearable devices 12 may be through a combination of wireless and wired communications. Furthermore, the communications between the administration device 16 and the wearable device 12 may be through a central server 46 or the communications between the administration device 16 and the wearable device 12 may be direct.

The administration devices 16 may also comprise administration device microphones 34, administration device speakers 36, and administrator device cameras 38 to allow for staff members 6 to communicate with the residents 2, as described below.

The wearable device processing unit 14 is preferably configured to detect certain events associated with the resident 2 wearing the wearable device 12 associated therewith. These events may include physiological events and geolocation events. For example, in one embodiment, the wearable device processing unit 14 is configured to detect a physiological event, such as a potential fall experienced by the resident 2. The wearable device 12 may comprise one or more sensors 22 that are configured to monitor movement or physiological events and that are operably connected to the wearable device processing unit 14. For example, where the sensors 22 are used to detect a potential fall, the sensors 22 may include accelerometer sensors, gyroscope sensors, or the like, which may be configured to monitor and relay information to the wearable device processing unit 14 regarding sudden or relative changes in orientation, position, and/or movement of the wearable device 12 that may be indicators of a potential fall by the resident 2 wearing the wearable device 12. The wearable device processing unit 14 is configured to receive information from the one or more sensors 22, and the wearable device processing unit 14 is further configured to generate an initial alert if the information from the one or more sensors 22 satisfy certain preset criteria that may be indicators of a potential fall.

In another embodiment, the physiological event may be a measure of a condition or a change in condition relating to a heart rate, heart rhythm, temperature, blood pressure, breathing rate, blood oxygenation level, blood sugar, or any other physiological or behavioral condition or change thereof with the resident 2, which may be detectable by the sensors 22 on the wearable device 12. For example, the sensors 22 may include one or more of the following: heart rate monitors, blood pressure monitors, thermometers, blood pressure readers, blood oxygen monitors, blood sugar monitors, or the like. The wearable device processing unit 14 may have one or more preset filters that may be selected by the resident 2 to identify a physiological event of significance that is desirably monitored by the wearable device processing unit 14. For example, the filters may also include a predetermined acceptable range of values for the physiological or behavioral condition being monitored. The sensors 22 are configured communicate information to the wearable device processing unit 14 regarding the physiological or behavioral condition being detected by the sensors 22. If the wearable device processing unit 14 detects that the information from the sensors 22 is outside of the range specified by the filters, the wearable device processing unit 14 is configured to generate an alert. When the alert is generated by the wearable device processing unit 14, the alert may include information on the type of physiological or behavioral event that resulted in the alert being generated.

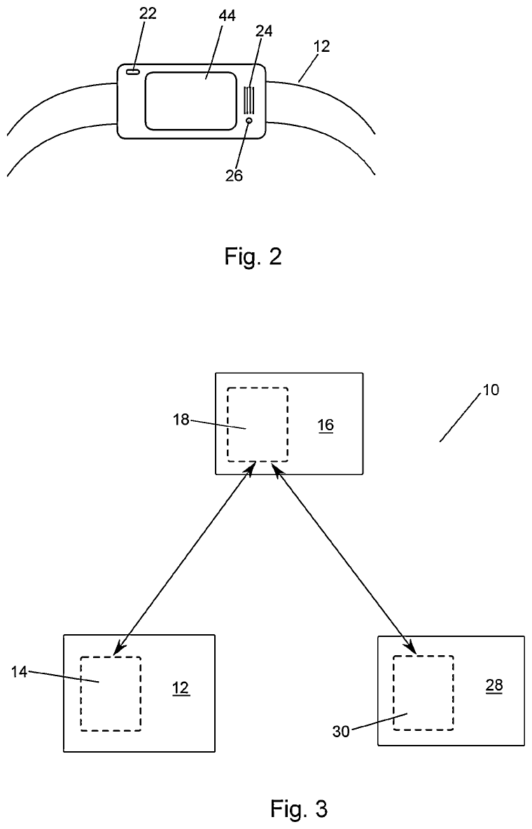

In one embodiment, illustrated in FIG. 2, the wearable device 12 may also comprise one or more wearable device speakers 24 that are operably connected to the wearable device processing unit 14. When the alert is generated by the wearable device processing unit 14, an appropriate signal is sent to the wearable device speakers 24 to sound a suitable audio message. For example, where the wearable device processing unit 14 has detected (through information from the sensors 22) that the resident 2 may have suffered a fall, the wearable device processing unit 14 may cause the audio message to be an audio prompt asking whether the resident 2 has suffered a fall. The wearable device 12 preferably comprises one or more wearable device microphones 26 that are configured to receive audio information and that are operably connected to the wearable device processing unit 14. The wearable device microphones 26 are configured to receive audio input from the resident 2 in response to the audio prompt.

The audio input received from the microphones 26 is sent to the wearable device processing unit 14, where the audio input is processed to determine whether the resident 2 responded to the audio prompt. If the wearable device processing unit 14 determines that the resident 2 responded to the audio prompt by saying that he or she is fine, then the wearable device processing unit 14 may take no further action. However, if the wearable device processing unit 14 determines that the resident 2 responded to the audio prompt by saying that he or she has fallen and/or requires assistance, or if the wearable device processing unit 14 determines that the resident 2 has not responded to the audio prompt, the wearable device processing unit 14 is configured to generate an event alert that is communicated to at least one of the administration devices 16. In one embodiment, the alert is transmitted by the wearable device 12 to the central server 46, which in turn relays the alert to an appropriate at least one of the administration devices 16. Preferably, the event alert is transmitted at least to the administration device 16 that is closest to the wearable device 12 or to the administration device 16 in the section of the congregate care facility 5 in which the resident 2 is associated. For example, in addition to associating the resident 2 with one or more of the wearable devices 12 and/or one of the rooms 4, the resident 2 may also be associated with one or more of the administration devices 16. The event alert may comprise information regarding the identity of the resident 2 associated with the wearable device 12, the room 4 associated with the wearable device 12, and the type of physiological event that triggered the event alert (e.g. a potential fall, irregular heart rate, etc.).

Alternatively, the wearable device 12 may comprise a wearable device interface 44, which may comprise a screen (e.g. a touchscreen) or one or more physical buttons. In response to the audio prompt asking whether the resident 2 has suffered a fall (or some other physiological event), the resident 2 is able to respond using the wearable device interface 44. This may be done, for example, by the resident 2 pushing a button (either a physical or virtual button) labelled “Yes” on the wearable device interface 44. The wearable device processing unit 14 determines, based on the response received by the wearable device interface 44, whether to generate an event alert for transmission to the one or more administration devices 16.

The system 10 further comprises room devices 28. Preferably, at least one of the room devices 28 is located in each of the rooms 4. Each of the room devices 28 may comprise a room device processing unit 30 that is configured to enable communications between the room device 28 and at least one of the administration devices 16, preferably through wireless communications. The administration device processing unit 18 is configured to enable communications between the administration device 16 and the room device 28, preferably through wireless communications. The wireless communication may be through Wi-Fi, ZigBee, Bluetooth, cellular networking, or the like. Each of the room devices 28 is associated with one of the residents 2. Therefore, preferably, each of the residents 2 is associated with at least one of the wearable devices 12, at least one of the rooms 4, and at least one of the room devices 28. The associations may be created as part of the registration process for the resident 2 and may be stored by the administration devices 16 or in the central server 46. Furthermore, the communications between the administration device 16 and the room device 28 may be through the central server 46 or the communications between the administration device 16 and the room device 28 may be direct.

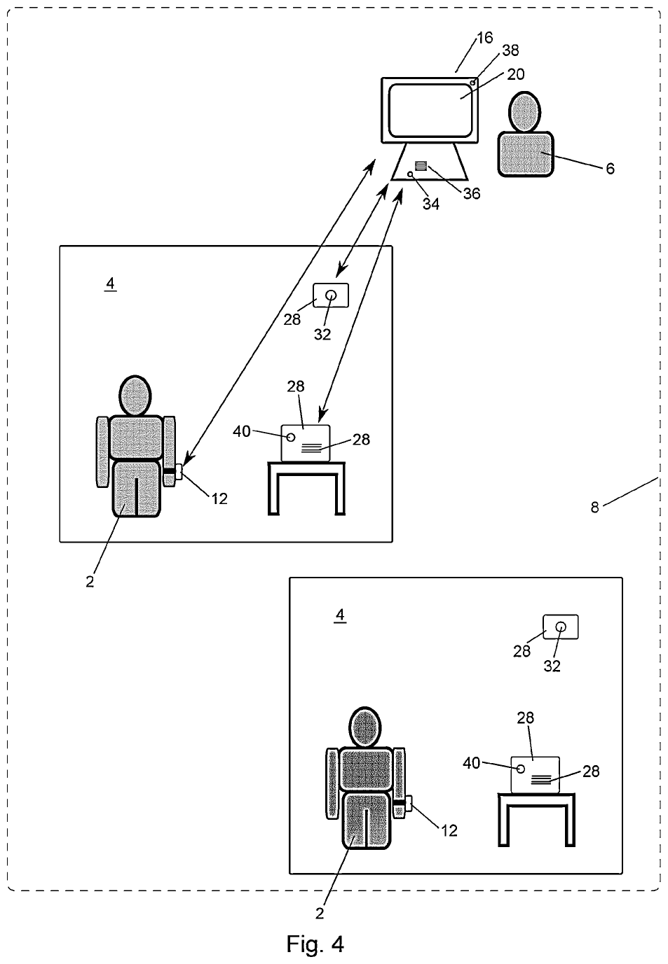

The room device 28 may comprises a room device camera 32 that is configured to capture video information regarding an interior of the room 4 in which the room device 28 is located. The room device camera 32 is configured to transfer the video information to at least one of the administration devices 16. The room device 28 may further comprise room device microphones 40 and room device speakers 42 that are configured to allow for communication between the resident 2 within the room 4 and at least one of the administration devices 16. In the embodiment shown in FIG. 1, the room device camera 32, the room device microphones 40, and the room device speakers 42 are located on the same physical unit (i.e. a single one of the room devices 28, such as a tablet or a laptop computer). However, in another embodiment (shown in FIG. 4), the room device camera 32, the room device microphones 40, and the room device speakers 42 may be located on separate ones of the room devices 28 that are all located within one of the rooms 4. For example, the camera 32 may be located on a first one of the room devices 28, while the room device microphones 40 and the room device speakers 42 are located on a second one of the room devices 28.

When one of the administration devices 16 receives the event alert from one of the wearable devices 12, the administration device processing unit 18 is configured to process the event alert and to display a suitable message on the administration device interface 20. The administration device processing unit 18 is configured to identify the resident 2, the room 4 associated with the wearable device 12 from which the event alert was received, and the physiological event triggering the event alert. The administration device processing unit 18 may also be configured to display this information on the administration device interface 20. In addition to appearing on the administration device interface 20, the administration device processing unit 18 may also cause the administration device speakers 36 to generate an audio message for the staff member 6.

The administration device processing unit 18 is further configured to communicate with the room device 28 in the room 4 associated with the wearable device 12 from which the event alert was received. The administration device processing unit 18 is configured to allow a staff member 6 proximate to the administrator device 16 to activate the room device camera 32 remotely from the administrator device 16 and begin receiving video information from the room device camera 32. As such, the room device processing unit 30 is configured to allow the administrator device 16 to remotely enable the room device camera 32. In one embodiment, the consent of the resident 2 may be first obtained. The video information is video information of an interior of the room, 4 which will allow observation of the situation of the resident 2 by the staff member 6 at the administration device 16 (e.g. at the nursing desk or front desk of the congregate care facility 5). This video information is transmitted to the administrator device 16, which may be configured to display the video information on the administration device interface 20.

The administrator device interface 20 allows the staff member 6 to view the video information of the interior of the room 4. From this video information, the staff member 6 may be able to observe whether the resident 4 has fallen or is otherwise in medical distress. In one embodiment, the administrator device interface 20 is configured to allow the staff member 6 to initiate a call (e.g., an audio and/or video call) between the administration device 16 and the room device 28. This may be accomplished through the use of the administration device microphones 34, the administration device speakers 36, and/or the administration device cameras 38 located on the administration device 16 and the use of the room device microphones 40, the room device speakers 42 and/or the room device camera 32 located on the room device 28. Alternatively, the room device 28 may also comprise an interface for communicating with the administration device 16.

After initiating a call between the administration device 16 and the room device 28, the staff member 6 may be able to try to communicate to the resident 2 to assess whether the resident 2 requires further assistance.

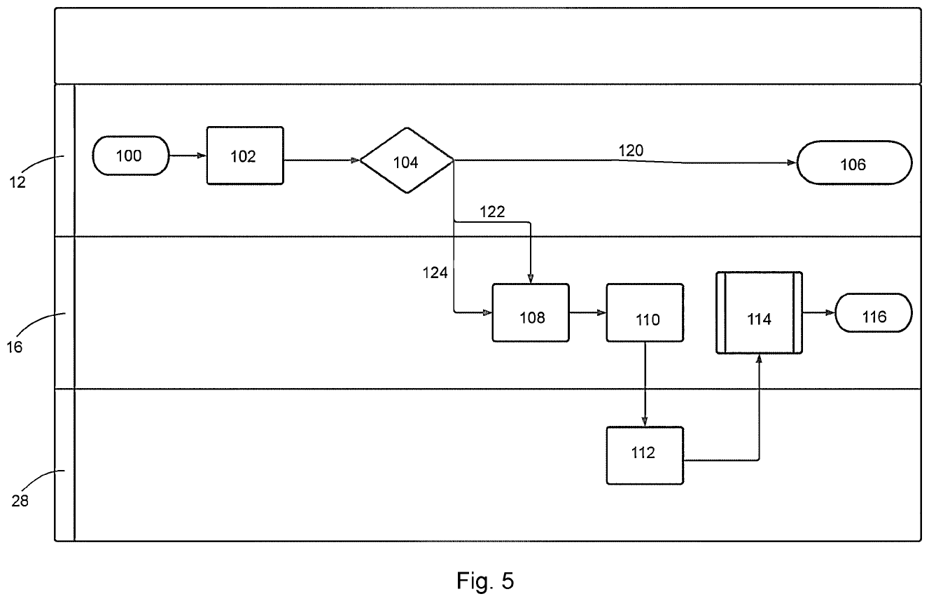

FIG. 5 depicts the process flow for the detection of a possible fall by the resident 2. The wearable device processing unit 14 may detect a possible fall by the resident 2 (step 100). This may be carried out by the sensors 22 on the wearable device 12 (e.g. accelerometer and/or gyroscope sensors) collecting information regarding relative linear and angular motion information of the wearable device 12 and transmitting the information to the wearable device processing unit 14. The wearable device processing unit 14 is configured to use the information to make a determination as to when a possible fall is detected.

When the wearable device processing unit 14 detects that a possible fall by the resident 2 has occurred, the wearable device processing unit 14 is configured to generate an audio alert (step 102). The audio alert is transmitted to the wearable device speakers 24 for broadcast. For example, the audio alert may be in the form of a verbal question, “Did you fall?” (step 104).

At this time, the resident 2 may be prompted by the wearable device interface 44 to either input “No” or “Yes”. If the resident 2 inputs “No” (step 120) through the wearable device interface 44, the wearable device processing unit 14 takes no further action (step 106). However, if the resident 2 inputs “Yes” through the wearable device interface 44 (step 122) or fails to enter an input through the wearable device interface 44 after a predetermined amount of time (step 124), an event alert is generated by the wearable device processing unit 14 and transmitted to one of the administration devices 16 (step 108). When the administration device 16 receives the event alert, an appropriate message or prompt is displayed on the administration device interface 20 for viewing by the staff member 6. The message or prompt may also indicate the particular one of the residents 2 associated with the wearable device 12 from which the event alert was received and the reason that the event alert was generated. The staff member 6 is then able to, using the administration device interface 20, initiate a call to the room device 28 associated with the resident 2 (step 110).

The room device 28 associated with the resident 2 is configured to accept the call from the administration device 16 (step 112). The staff member 6 and the resident 2 are then able to communicate, using the administration device microphones, speakers, and camera 34, 36, 38 and the room device microphones, speakers, and cameras 40, 42, 32, respectively (step 114). The staff member 6 may be able to assess whether further assistance for the resident 2 is required. For example, if the staff member 6 determines that no further assistance is required, the staff member 6 may clear the event alert using the administration device interface 20 (step 116).

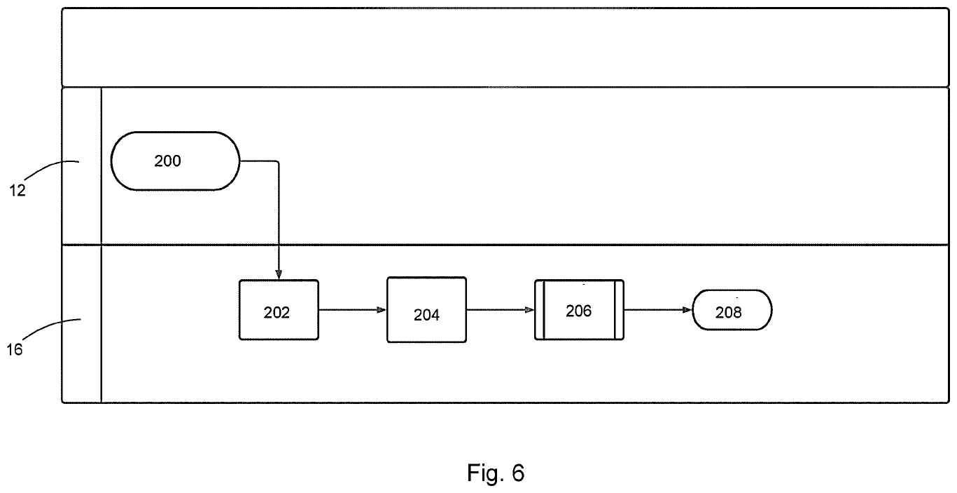

In another embodiment, referring to FIG. 6, in addition to detecting possible physiological events for one of the residents 2, the system 10 may also detect a geolocation event when one of the residents 2 is outside of a predefined position (e.g., outside of the congregate care facility 5, outside of a particular floor, outside of a particular area, etc.). The geolocation event could be based on the movement of the wearable device 12 to a location which is restricted or prohibited to the resident 2. For example, the resident 2 wearing the wearable device 12 may be experiencing early signs of dementia or confusion. It may be desirable the staff members 6 of the facility be notified if the resident 2 wanders outside of a safety-controlled area.

In this embodiment, the wearable device 12 is preferably associated with a predefined geographical area 8 (in addition to being associated with one of the residents 2 and a room 4). The predefined geographical area 8 may be any defined physical area. For example, it may be the room 4 associated with the resident 2. Alternatively, it may be a floor of the congregate care facility 5 or it may be the congregate care facility 5 itself. The sensors 22 on the wearable device 12 are further configured to determine a location of the wearable device 12. This may be done using global positioning system (GPS) sensors or by using received signal strength indicator (RSSI). The location information is used by the wearable device processing unit 14 to determine whether the wearable device 12 (and thus the resident 2) is located outside of the predefined geographical area 8 associated with the resident 2, as shown in FIG. 4.

Preferably, the wearable device 12 will periodically transmit location information (regarding the present location of the wearable device 12) to the administration device 16. This may be done through the central server 46 or this may be done directly from the wearable device 12 to the administration device 16. When the communication is done through the central server 46, the central server 46 is configured to determine the appropriate one or ones of the administration devices 16 to which the location information is sent. Preferably, the location information is transmitted at least to the administration device 16 that is closest to the wearable device 12 or to the administration device 16 to which the resident 2 is associated.

If the wearable device processing unit 14 determines that the wearable device 12 is located outside of the predefined geographical area 8 associated with the wearable device 12, the wearable device processing unit 14 is configured to generate a location alert and transmit it to the administration device 16 (either directly or through the central server 46). When the communication is done through the central server 46, the central server 46 is configured to determine the appropriate one or ones of the administration devices 16 to which the location alert is sent. Preferably, the location alert is transmitted at least to the administration device 16 that is closest to the wearable device 12 or to the administration device 16 to which the resident 2 is associated.

Upon receipt of the location alert from the wearable device 12 by the administration device 16, the administration device processing unit 18 will cause the administration device interface 20 to display a suitable message. In addition, the administration device processing unit 18 may also cause the administration device interface 20 to display the last location of the wearable device 12.

Alternatively, instead of the wearable device processing unit 14 generating the location alert when it determines that the wearable device 12 is located outside of the predefined geographical area 8 associated with the resident 2, the administration device processing unit 18 may be configured to generate a location alert if the administration device 16 fails to receive the location information from the wearable device 12 after a predetermined amount of time. This will allow the location alert to be generated even if the wearable device 12 is no longer within wireless communications range of the administration device 16.

FIG. 6 depicts the process flow for the embodiment. The wearable device processing unit 14 may detect if the wearable device 12 is outside of the predefined geographical area 8 (step 200). This may be carried out by the sensors 22 on the wearable device 12 (e.g. GPS sensors) collecting information regarding a location of the wearable device 12 and transmitting the information to the wearable device processing unit 14. The wearable device processing unit 14 is configured to use the information to make a determination as to when the wearable device 12 is outside of the predefined geographical area 8.

When the wearable device processing unit 14 detects that the wearable device 12 is outside of the predefined geographical area 8, the wearable device processing unit 14 is configured to transmit the location alert to one of the administration devices 16 (step 202). The wearable device processing unit 14 may also be configured to periodically transmit location information to one of the administration devices 16, the location information comprising a current location of the wearable device 12 (as detected by the sensors 22). When the location alert is received by the administration device 16, an appropriate message or prompt is displayed on the administration device interface 20 for viewing by the staff member 6. The message or prompt may also indicate the particular one of the residents 2 associated with the wearable device 12 from which the location alert was received and the last transmitted location of the wearable device 12 (step 204).

The staff member 6 is able to view the identity of the resident 2 associated with the wearable device 12 from which the location alert was received and the last transmitted location of the wearable device 12. Based on this information, the staff member 6 may determine the appropriate action (if any) to be taken (step 206). After the appropriate action has been taken, the staff member 6 is able to clear the location alert using the administration device interface 20 (step 208).

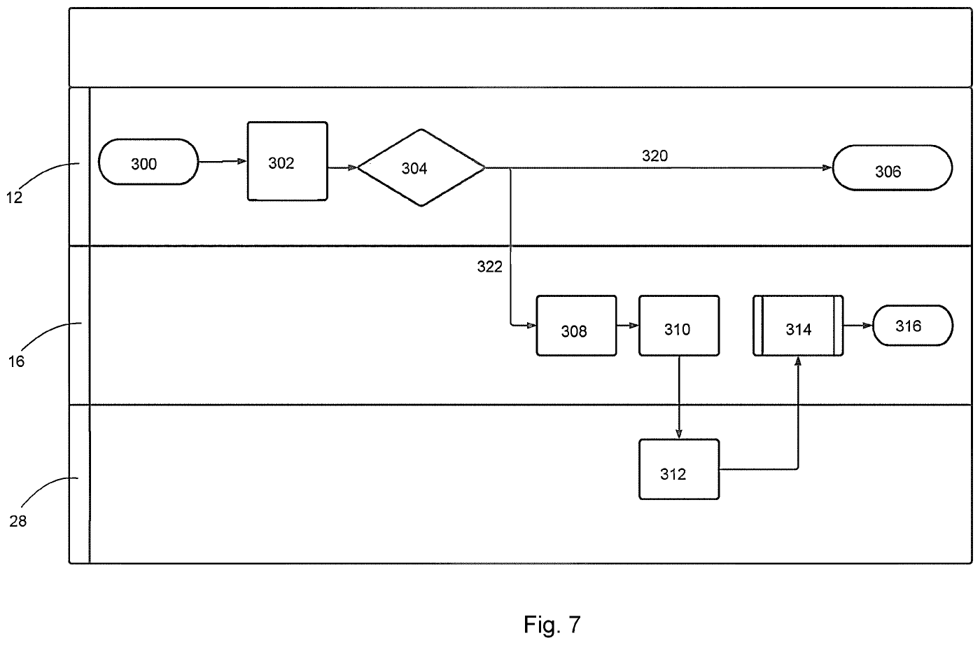

In another embodiment, referring to FIG. 7, the wearable device interface 44 is configured to allow the resident 2 to communicate with the administration device 16. For example, the resident 2 may use the wearable device interface 44 to signal that the resident 2 is in need of assistance (e.g., by pressing on the wearable device interface 44 a suitable icon or by pressing a physical button). This will cause the wearable device processing unit 14 to generate a help alert and to transmit it to the administration device 16 (either directly or through the central server 46). When the communication is done through the central server 46, the central server 46 is configured to determine the appropriate one or ones of the administration devices 16 to which the location information is sent. Preferably, the help alert is transmitted at least to the administration device 16 that is closest to the wearable device 12 or to the administration device 16 to which the resident 2 is associated.

The administration device processing unit 18, upon the administration device 16 receiving the help alert, may cause the administration device interface 20 to display a suitable message for viewing by the staff member 6. The staff member 6, using the administration device interface 20, may initiate a call (e.g., an audio and/or video call) between the administration device 16 and the room device 28 that is located in the room 4 associated with the wearable device 12. After initiating a call between the administration device 16 and the room device 28, the staff member 6 may be able to try to communicate to the resident 2 to assess whether the resident 2 requires medical assistance.

FIG. 7 depicts the process flow for the embodiment. The resident 2 is able to initialize the wearable device interface 44 and can provide an appropriate input to the wearable device interface 44 (step 300), such as pressing a button labelled “Help”. Upon receiving such an input, the wearable device processing unit 14 is configured to prompt the resident 2 to confirm that the resident 2 wishes to seek assistance (step 302). For example, this may include the wearable device processing unit 14 causing the wearable device interface 44 to display a message prompting the resident 2 to push and hold a button on the wearable device interface 44 (step 304).

If the resident 2 does not confirm the prompt (step 320), the wearable device processing unit 14 determines that no further action is required (step 306). If, however, the resident 2 does confirm the prompt (e.g. by pushing and holding a button on the wearable device interface 44) (step 322), the wearable device processing unit 14 is configured to generate the help alert and transmit it to one of the administration devices 16 (step 308).

When the help alert is received by the administration device 16, an appropriate message or prompt is displayed on the administration device interface 20 for viewing by the staff member 6. The message or prompt may also indicate the particular one of the residents 2 associated with the wearable device 12 from which the help alert was received.

The staff member 6 is able to view the identity of the resident 2 associated with the wearable device 12 from which the help alert was received. The staff member 6 is then able to, using the administration device interface 20, initiate a call to the room device 28 associated with the resident 2 (step 310).

The room device 28 associated with the resident 2 is configured to accept the call from the administration device 16 (step 312). The staff member 6 and the resident 2 are then able to communicate, using the administration device microphones, speakers, and camera 34, 36, 38 and the room device microphones, speakers, and cameras 40, 42, 32, respectively (step 314). The staff member 6 may be able to assess whether further assistance for the resident 2 is required. For example, if the staff member 6 determines that no further assistance is required, the staff member 6 may clear the event alert using the administration device interface 20 (step 316).

In one embodiment, the system 10 may be implemented using software executing on various hardware platforms. For example, the wearable device 12 may be implemented by executing a mobile application on a smartwatch device. The mobile application is able to access various components on the smartwatch device, including the sensors 22 and the wearable device speakers and microphones 24, 26. Similarly, the room device 28 may be implemented by executing a mobile application on a tablet computer. The mobile application is able to access various components on the tablet computer, including the room device camera 32 and the room device microphones and speakers 40, 42.

The administration devices 16 may comprise a single one of the administration devices 16 that is responsible for receiving communications from all of the room devices 28 in the facility. Alternatively, there may be a number of the administration devices 16 located at various locations within the facility (e.g. one on each floor of the facility, etc.). Furthermore, the administration devices 16 may be implemented as a software platform executing on a central computer. The central computer may be configured to relay information (e.g. video information, voice communications, etc.) to other computers or devices operated by the staff members 6. For example, the staff members 6 may use mobile devices to access the central computer in order to access video information or to receive event alerts.

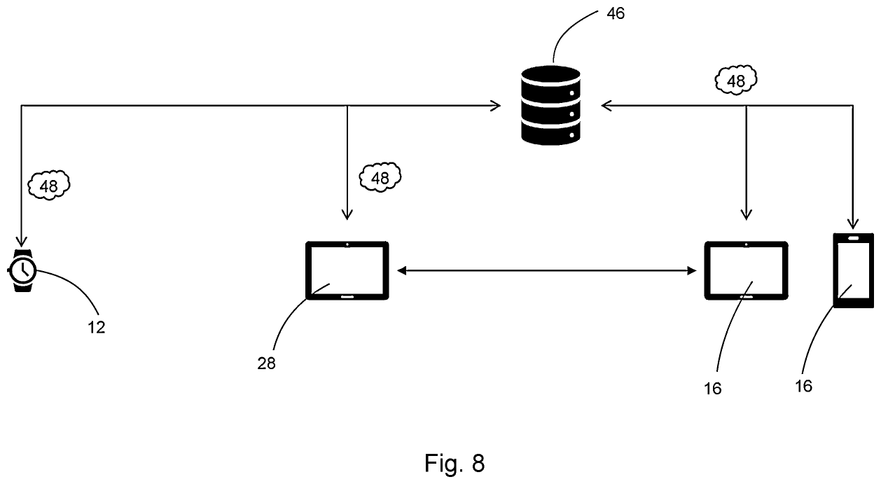

Referring to FIG. 8, in another embodiment, the system 10 may utilizes the central server 46 to coordinate the activities among the wearable devices 12, the administration devices 16, and the room devices 28. In this embodiment, the wearable devices 12, the administration devices 16, and the room devices 28 are configured to communicate with the server 12. Such communications may be wired or wireless communications and may be through a network 48. For example, the wearable device 12 may be configured to transmit to the server 46 various alerts, including event alerts, location alerts, and help alerts. The server 46, in turn, is configured to transmit the alerts (including the event alerts, the location alerts, and the help alerts) to one or more of the administration devices 16.

The server 46 is also configured to store information associated with each of the residents 2, including information regarding the rooms 4, the wearable devices 12, the room devices 28, the administration devices 16, and/or the predefined geographical area 8 associated with each of the residents 2.

The administration devices 16 are configured to receive from the server 46 the alerts (including the event alerts, the location alerts, and the help alerts). The administration devices 16 may also transmit information to the server 46 regarding the residents 2 (including the administration device(s) 16, the room(s) 4, the wearable device(s) 12, and the room device(s) 28 associated therewith) for recording and updating by the server 46. For example, the administration devices 16 may be used during the registration process to create the associations between the resident 2 and the rooms 4, the wearable devices 12, and the room devices 28.

The room devices 28 and the administration devices 16 may be configured to communicate in a peer-to-peer fashion, such as when a call is connected between the two.

The system 10 allows for the administration device 16 to receive communications in the form of alerts from individual ones of the wearable devices 12. The system 10 further allows the administration device 16 to communicate with the appropriate ones of the room devices 28 individually in response to receiving alerts from the wearable devices 12.

It will be appreciated by those skilled in the art that the preferred embodiments have been described in some detail but that certain modifications may be practiced without departing from the principles of the invention.

1. A system for monitoring residents in a congregate care facility comprising:
one or more administration devices:
one or more wearable devices, each of the one or more wearable devices removably attached to and associated with one of the residents and configured to determine an alert associated with the resident and to communicate the alert to at least one of the administration devices:
one or more room devices, wherein each of the room devices is associated with one of the residents, and wherein for the one or more room devices associated with one of the residents, at least one of the room devices comprises a camera configured to communicate with the administration device, and at least one of the room devices is configured to allow for voice communications with the administration device:
wherein the administration device is configured to:
i) receive the alert from the wearable device;
ii) identify the resident and the room device associated with the wearable device:
iii) activate the camera of the room device associated with the resident: and
iv) allow for communication between the room device associated with the resident and the administration device.

2. The system of claim 1, wherein the alert is a physiological alert.

3. The system of claim 2, wherein the physiological alert may be selected from one or more of the following: physical fall, blood pressure, heart rhythm, body temperature, heart rate, oxygenation level, and blood sugar.

4. The system of claim 3, wherein each of the one or more wearable devices comprises one or more sensors configured to detect information relating to the physiological alert.

5. The system of claim 4, wherein each of the one or more wearable devices is configured to communicate the alert when the information relating to the physiological alert detected by the one or more sensors fall outside of a predetermined range of values.

6. The system of claim 1, further comprising a server, wherein the server is configured to receive the alert from one of the wearable devices and wherein the server is further configured to determine an appropriate ones of the administration devices to which to send the alert.

7. The system of claim 6, wherein the determination of the appropriate ones of the administration devices to which to send the alert is determined, at least in part, by determining the administration device that is closest to the wearable device from which the alert was communicated.

8. The system of claim 6, wherein each of the administration devices is associated with one or more of the residents.

9. The system of claim 8, wherein the determination of the appropriate one of the administration devices to which to send the alert is determined, at least in part, by determining the administration devices associated with the resident whose wearable device from which the alert was communicated.

10. A system for monitoring residents in a congregate care facility comprising:
one or more administration devices:
one or more wearable devices, each of the one or more wearable devices removably attached to and associated with one of the residents and configured to receive a request for assistance from with the resident and to communicate the request for assistance to at least one of the administration devices:
one or more room devices, wherein each of the room devices is associated with one of the residents, and wherein for the one or more room devices associated with one of the residents, at least one of the room devices comprises a camera configured to communicate with the administration device, and at least one of the room devices is configured to allow for voice communications with the administration device:
wherein the administration device is configured to:
i) receive the request for assistance from the wearable device:
ii) identify the resident and the room device associated with the wearable device:
iii) activate the camera of the room device associated with the resident: and
iv) allow for communication between the room device associated with the resident and the administration device.

11. The system of claim 10, further comprising a server, wherein the server is configured to receive the request for assistance from one of the wearable devices and wherein the server is further configured to determine an appropriate ones of the administration devices to which to send the request for assistance.

12. The system of claim 11, wherein the determination of the appropriate ones of the administration devices to which to send the request for assistance is determined, at least in part, by determining the administration device that is closest to the wearable device from which the request for assistance was communicated.

13. The system of claim 11, wherein each of the administration devices is associated with one or more of the residents.

14. The system of claim 13, wherein the determination of the appropriate one of the administration devices to which to send the request for assistance is determined, at least in part, by determining the administration devices associated with the resident whose wearable device from which the request for assistance was communicated.

15. A system for monitoring residents in a congregate care facility comprising:
a central server:
one or more administration devices in communications with the central server; and
one or more wearable devices in communications with the central server, each of the one or more wearable devices removably attached to and associated with one of the residents and configured to determine a location of the wearable device and to communicate the location to the central server from time to time:
wherein the wearable device is configured to generate an alert when the wearable device determines that it is outside of a predefined geographical area, the alert being transmitted to the central server; and
wherein the central server is configured to transmit the alert and the location of the wearable device from time to time to one or more of the administration devices.

16. The system of claim 15, wherein the central server is configured to transmit the alert and the location of the wearable device from time to time to the administration device that is closest to the wearable device.

17. The system of claim 15, wherein each of the administration devices is associated with one or more of the residents.

18. The system of claim 17, wherein the central server is configured to transmit the alert and the location of the wearable device from time to time to the administration device that is associated with the resident whose wearable device from which the alert was communicated.
